(19) United States
(12) Patent Application Publication
(10) Pub. No.: US 2016/0331227 A1
(43) Pub. Date: Nov. 17, 2016

(54) EXTENDED DURATION OPTICAL COHERENCE TOMOGRAPHY (OCT) SYSTEM

(71) Applicants: UNIVERSITY OF SOUTHERN CALIFORNIA, Los Angeles, CA (US); CALIFORNIA INSTITUTE OF TECHNOLOGY, Pasadena, CA (US)

(72) Inventors: Jeffrey P. Fingler, Orange, CA (US); Scott E. Fraser, Glendale, CA (US)

(73) Assignees: UNIVERSITY OF SOUTHERN CALIFORNIA, Los Angeles, CA (US); CALIFORNIA INSTITUTE OF TECHNOLOGY, Pasadena, CA (US)

(21) Appl. No.: 15/106,949

(22) PCT Filed: Mar. 4, 2015
(86) PCT No.: PCT/US15/18789

§ 371 Date: Jun. 21, 2016

Related U.S. Application Data
(63) Continuation of application No. PCT/US2015/018637, filed on Mar. 4, 2015.
(60) Provisional application No. 61/947,831, filed on Mar. 4, 2014.
(60) Provisional application No. 62/112,538, filed on Feb. 5, 2015.

(51) Int. Cl. A61B 3/10; A61B 3/113; A61B 3/12; A61B 3/00

(52) U.S. Cl. CPC A61B 3/102; A61B 3/0083; A61B 3/0091; A61B 3/113; A61B 3/0025; A61B 3/1241

(57) ABSTRACT

This disclosure relates to the field of Optical Coherence Tomography (OCT). This disclosure particularly relates to methods and systems for providing larger field of view OCT images. This disclosure also particularly relates to methods and systems for OCT angiography. These systems may allow OCT scanning for an extended duration and generation of large field OCT images suitable for the OCT angiography.

### CROSS-REFERENCE TO RELATED APPLICATIONS

This application is based upon and claims priority to U.S. provisional patent application 61/947,831, entitled “Differential Coupling Optical Coherence Tomography (OCT) Imaging and Extended Duration OCT Angiography Imaging System,” filed Mar. 4, 2014, attorney docket number 028080-0988; and U.S. provisional patent application 62/112,538, entitled “Extended Duration Optical Coherence Tomography (OCT),” filed Feb. 5, 2015, attorney docket number 064693-0315. This application is also based upon and claims priority to Patent Cooperation Treaty (PCT) application No. PCT/US15/18637, entitled “Optical Coherence Tomography System for Health Characterization of an Eye,” filed Mar. 4, 2015, attorney docket number 064693-0316. The entire contents of these provisional patent applications and PCT application are incorporated herein by reference.

### STATEMENT REGARDING FEDERALLY SPONSORED RESEARCH

This invention was made with government support under Grant No. NIH STTR 1 R41 EY021054 awarded by National Institutes of Health (NIH). The government has certain rights in the invention.

### BACKGROUND

1. Technical Field

This disclosure relates to the field of Optical Coherence Tomography (OCT). This disclosure particularly relates to methods and systems for providing larger field of view OCT images. This disclosure also particularly relates to methods and systems for angiography.

2. Description Of Related Art

Optical coherence tomography (OCT) has become an important clinical imaging tool, since its introduction in 1991. For a background of OCT technology, see, for example, Drexler and Fujimoto et al. “Optical Coherence Technology: Technology and Applications” Springer, Heidelberg, Germany, 2008. This book is incorporated herein by reference in its entirety. OCT is based on an optical measurement technique known as low-coherence interferometry. OCT performs high resolution, cross-sectional imaging of internal microstructure of a physical object by directing a light beam to the physical object, and then measuring and analyzing magnitude and time delay of backscattered light.

A cross-sectional image is generated by performing multiple axial measurements of time delay (axial scans or A-scans) and scanning the incident optical beam transversely. This produces a two-dimensional data set of A-scans, which represents the optical backscattering in a cross-sectional plane through the physical object (i.e. B-scans). Three-dimensional, volumetric data sets can be generated by acquiring sequential cross-sectional images by scanning the incident optical beam in a raster pattern (three-dimensional OCT or 3D-OCT). This technique yields internal microstructural images of the physical objects with very fine details. For example, pathology of a tissue can effectively be imaged in situ and in real time with resolutions smaller than 15 micrometers.

Several types of OCT systems and methods have been developed, for example, Time-domain OCT (TD-OCT) and Fourier-domain OCT (FD-OCT). Use of FD-OCT enables high-resolution imaging of retinal morphology that is nearly comparable to histologic analysis. Examples of FD-OCT technologies include Spectral-domain OCT (SD-OCT) and Swept-source OCT (SS-OCT).

OCT may be used for identification of common retinovascular diseases, such as age-related macular degeneration (AMD), diabetic retinopathy (DR), and retinovascular occlusions. However, despite the rapid evolution of OCT imaging, current OCT technology may not provide adequate visualization of retinal and choroidal microvasculature. Thus, clinicians are often compelled to order both OCT and fluorescein angiography (FA) in patients with the retinovascular diseases.

Since their introduction more than 50 years ago, fluorescein angiography (FA) and indocyanine green angiography (ICGA) have been used for retinovascular imaging. This method typically involves an injection of fluorescent dye into the blood stream, and the perfusion of the dye into the retinal and choroidal blood vessels is observed optically on the fundus. An estimated 1 million FA studies are performed annually in the United States. Although FA has obvious value in revealing fine details of the microvasculature, it may require an intravenous injection and a skilled photographer and can be time-consuming. Minor side effects such as nausea, vomiting, and multiple needle sticks in patients with challenging venous access are not uncommon. Because fluorescein leaks readily through the fenestrations of the choriocapillaris, it may not be suitable for showing the anatomy of this important vascular layer that supplies the outer retina. ICGA provides improved visualization of choroidal anatomy because this dye is more extensively protein bound than fluorescein and may not leak into the extravascular space as readily. Furthermore, it fluoresces at a longer wavelength than fluorescein and imaging can take place through pigment and thin layers of blood. Nevertheless, ICGA may fail to depict the fine anatomic structure of the choriocapillaris.

There has been increased interest in using data generated during FD-OCT imaging to generate angiographic images of the fundus. These angiograms can be implemented noninvasively without injection of fluorescent dye.

Recently, phase-variance OCT (PV-OCT) has been introduced to image retinal microvasculature. See, for example, Fingler et al. “Dynamic Motion Contrast and Transverse Flow Estimation Using Optical Coherence Tomography” U.S. Pat. No. 7,995,814; Fingler et al. “Dynamic Motion Contrast and Transverse Flow Estimation Using Optical Coherence Tomography” U.S. Pat. No. 8,369,594; Fingler et al. “Mobility and transverse flow visualization using phase variance contrast with spectral domain optical coherence tomography” Opt. Express 2007; 15:12636-53; Fingler et al. “Phase-contrast OCT imaging of transverse flows in the mouse retina and choroid” Invest Ophthalmol. Vis. Sci. 2008;49:5055-9; Fingler et al. “Volumetric microvascular imaging of human retina using optical coherence tomography with a novel motion contrast technique” Opt. Express 2009;17:22190-200; Kim et al. “In vivo volumetric imaging of human retinal circulation with phase-variance optical coherence tomography” Biomed Opt Express [serial online] 2011; 2:1504-13; Kim et al. “Noninvasive imaging of the foveal avascular zone with high-speed, phase-variance optical coherence tomography” Invest. Ophthalmol. Vis. Sci. 2012; 53:85-92; and Kim et al. “Optical imaging of the chorioretinal vasculature in the living human eye” PNAS, Aug. 27, 2013, vol. 110, no. 35, 14354-14359. All these publications and patent disclosures are incorporated herein by reference in their entirety.

PV-OCT uses software processing of data normally acquired, but not used, during FD-OCT imaging. With a different scanning protocol than found in commercial instruments, PV-OCT identifies regions of motion between consecutive B-scans that are contrasted with less mobile regions. In the retina and choroid, the regions with motion correspond to the vasculature; these vessels are readily differentiated from other retinal tissues that are relatively static.

An alternative method to acquire images of the retinal vasculature is Doppler OCT, which measures the change in scatterer position between successive depth scans and uses this information to calculate the flow component parallel to the imaging direction (called axial flow). Doppler OCT has been used to image large axial flow in the retina, but without dedicated scanning protocols this technique may be limited in cases of slow flow or flow oriented transverse to the imaging direction. Because this technique depends on measuring motion changes between successive depth scans, as imaging speed improvements continue for FD-OCT systems, the scatterers may have less time to move between measurements and the slowest motions may become obscured by noise. This can further reduce the visualization capabilities of typical Doppler OCT techniques.

In contrast, PV-OCT may be able to achieve the same time separations between phase measurements with increased FD-OCT imaging speeds, maintaining the demonstrated ability to visualize fast blood vessel and slow microvascular flow independently of vessel orientation.

Several groups in recent years have developed OCT imaging methods to push beyond conventional Doppler OCT imaging limitations. Some approaches involve increasing the flow contrast through hardware modifications of FD-OCT machines, such as in 2-beam scanning, or producing a heterodyne frequency for extracting flow components. Other investigators have used nonconventional scanning patterns or repeated B-scan acquisitions, such as used in PV-OCT to increase the time separation between phase measurements and enhance Doppler flow contrast of microvascular flow. In addition to phase-based contrast techniques to visualize vasculature, intensity-based visualization of microvasculature has been developed for OCT using segmentation, speckle-based temporal changes, decorrelation-based techniques, and contrast based on both phase and intensity changes. Each of these methods has varying capabilities in regard to microvascular visualization, noise levels, and artifacts while imaging retinal tissues undergoing typical motion during acquisition. Some of the noise and artifact limitations can be overcome with selective segmentation of the volumetric data or increased statistics through longer imaging times, but further analysis may be required to be able to compare all of the visualization capabilities from all these different systems.

For further description of OCT methods and systems, and their applications, for example, see: Schwartz et al. “Phase-Variance Optical Coherence Tomography: A Technique for Noninvasive Angiography” American Academy of Ophthalmology, Volume 121, Issue 1, January 2014, Pages 180-187; Sharma et al. “Data Acquisition Methods for Reduced Motion Artifacts and Applications in OCT Angiography” U.S. Pat. No. 8,857,988; Narasimha-Iyer et al. “Systems and Methods for Improved Acquisition of Ophthalmic Optical Coherence Tomography Data” U.S. Patent Application Publication No. 2014/0268046. All these publications and patent disclosures are incorporated herein in their entirety.

### SUMMARY

This disclosure relates to the field of Optical Coherence Tomography (OCT). This disclosure particularly relates to methods and systems for providing larger field of view OCT images. This disclosure also particularly relates to methods and systems for OCT angiography. This disclosure further relates to methods for health characterization of an eye by OCT angiography.

This disclosure relates to an extended duration optical coherence tomography (OCT) system for health characterization of an eye of a human. This system may comprise an OCT data acquisition system and a gravity-assisted head stabilization system. The OCT data acquisition system may have configuration that (a) scans tissue of an eye of a subject, which has a surface and a depth, with a beam of light that has a beam width and a direction; (b) acquires OCT signals from the scan; (c) forms at least one B-scan cluster set using the acquired OCT signals such that each B-scan cluster set includes at least two B-scan clusters; each B-scan cluster includes at least two B-scans; and each B-scan includes at least two A-scans; and (d) calculates OCT data using the at least one B-scan cluster set.

The gravity-assisted head stabilization system may provide stability for the subject's head and the eye when the OCT data acquisition system scans the tissue. The gravity-assisted head stabilization system may comprise a headrest. This headrest may have a configuration such that when the subject rests his/her head on the headrest, an axis passing through the subject's cranial vertex and that is parallel to the subject's coronal plane (“vertex axis”) does not become parallel to an axis vertical to earth's surface (“vertical axis”). In other words, for this configuration, the vertex axis is not perpendicular to surface of the earth at the subject's location. That is, in this configuration, the angle between the vertical axis and the vertex axis (“tilt angle”) may not be zero or may not substantially close to zero. The tilt angle may be at least 5 degrees or −5 degrees.

The headrest may also have a configuration such that when the subject rests his/her head on the headrest, the tilt angle may be in the range of 10 degrees to 90 degrees. The tilt angle may also be in the range of −10 degrees to −90 degrees. The tilt angle may also be in the range of 80 degrees to 90 degrees. The tilt angle may also be in the range of −80 degrees to −90 degrees.

The OCT data acquisition system may comprise a physical object arm. The physical object arm may mechanically be affixed to the headrest.

The gravity-assisted head stabilization system may further comprise an inclined chair system, a horizontal table system, or a combination thereof.

The extended duration OCT system may further comprise a dynamic fixation target system that stabilizes movement of the subject's eye. The dynamic fixation target system may comprise at least one fixation target.

The extended duration OCT system may also further comprise a system that automatically detects blinking of the subject and compensates for effects of blinking on the calculated OCT data. This system may further have a configuration that automatically stops acquisition of the OCT signals at onset of a blinking. This system may also further have a configuration that automatically starts acquisition of the OCT signals after a blinking. This system may also further have a configuration that automatically detects blinking by detecting a strong instantaneous decrease or increase in intensity of the acquired OCT signals. This system may also further have a configuration that automatically detects blinking by using the calculated OCT data.

The extended duration OCT system may also further comprise a camera; and the system may further have a configuration that uses images provided by the camera to detect blinking.

The extended duration OCT system may also further comprise an eye motion tracking system and uses information provided by this tracking system to minimize effects of the eye motion on the calculated OCT data.

The extended duration OCT system may further have a configuration that blocks light to a non-imaged eye.

The extended duration OCT system may also further have a configuration that calculates OCT data using motion occurring within the eye tissue and at least one B-scan cluster set formed by the OCT data acquisition system. In this OCT system, the OCT data may be calculated by using variations of intensity or phase of the OCT signals to provide contrast. In this OCT system, the OCT data may be calculated by using variations of intensity or phase of the OCT signals caused by flow, speckle, or decorrelation of an OCT signal within the OCT signals that may be caused by eye tissue motion or blood flow in blood vessels of the eye tissue.

Any combination of above features, products and methods is within the scope of the instant disclosure.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates a generalized OCT system.

FIG. 2 schematically illustrates an example of a scanning configuration for the OCT system illustrated in FIG. 1.

FIG. 3 schematically illustrates a sagittal view of an exemplary left human eye.

FIG. 4 schematically illustrates cross sectional layers of an exemplary retina.

FIG. 5 shows a cross-sectional (2D) OCT image of the fovea region of an exemplary retina.

FIG. 6 shows (A) an exemplary en-face OCT angiography image of an exemplary retinal vasculature around optic disc, (B) a magnified region of the OCT image of (A).

FIG. 7 schematically illustrates visual field of a fundus of an exemplary left eye of a healthy human.

FIG. 8 shows an example of an intensity distribution of a beam of light, transverse to the propagation direction.

FIG. 9 schematically illustrates four B-scans, two B-scan clusters, and one B-scan cluster set by way of example that may be used for the calculation of an OCT angiography data.

FIG. 10 schematically illustrates a subject's eye and head alignment with respect to an axis vertical to the earth' surface.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

This disclosure relates to an extended duration OCT system. The extended duration OCT system may comprise any interferometer that have optical designs, such as Michelson interferometer, Mach-Zehnder interferometer, Gires-Tournois interferometer, common-path based designs, or other interferometer architectures. The sample and reference arms in the interferometer may include any type of optics, for example bulk-optics, fiber-optics, hybrid bulk-optic systems, or the like.

This disclosure relates to the field of Optical Coherence Tomography (OCT). This disclosure particularly relates to methods and systems for providing larger field of view OCT images. This disclosure also particularly relates to methods and systems for OCT angiography. This disclosure further relates to methods for health characterization of an eye by OCT angiography.

The extended duration OCT system may also include any OCT system. Examples of the OCT systems may include Time-domain OCT (TD-OCT) and Fourier-domain, or Frequency-domain, OCT (FD-OCT). Examples of the FD-OCT may include Spectral-domain OCT (SD-OCT), Swept Source OCT (SS-OCT), and Optical frequency domain Imaging (OFDI).

The OCT system may use any OCT approach that identifies and/or visualizes regions of motion (“OCT angiography”). The OCT angiography may use motion occurring within the physical object to identify and/or visualize regions with improved contrast based on variations in the intensity and/or phase of the OCT signal. For example, these variations are caused by flow, speckle or decorrelation of the OCT signal caused by eye motion or flow in blood vessels. For example, variation of OCT signals caused by blood flow in blood vessels may be used by OCT to identify and/or visualize retinal or choroidal vasculature in the eye through the OCT angiography. As a result, structures and functions can be visualized that cannot be identified through a typical OCT system. For example, choriocapillaris may become visible by using the OCT angiography.

Examples of the OCT angiography may include Phase Variance OCT (PV-OCT), Phase Contrast OCT (PC-OCT), Intensity/Speckle Variance OCT (IV-OCT), Doppler OCT (D-OCT), Power of Doppler Shift OCT (PDS-OCT), Split Spectrum Amplitude Decorrelation Analysis (SSADA), Optical Micro-angiography (OMAG), Correlation Mapping OCT (cmOCT), and the like.

Examples of the PV-OCT are disclosed by Fingler et al. “Dynamic Motion Contrast and Transverse Flow Estimation Using Optical Coherence Tomography” U.S. Pat. No. 7,995,814; Fingler et al. “Dynamic Motion Contrast and Transverse Flow Estimation Using Optical Coherence Tomography” U.S. Pat. No. 8,369,594; Fingler et al. “Mobility and transverse flow visualization using phase variance contrast with spectral domain optical coherence tomography” Opt. Express [serial online] 2007; 15:12636-53; examples of the Speckle Variance OCT are disclosed by Mariampillai et al. “Speckle variance detection of microvasculature using swept-source optical coherence tomography,” Opt. Lett. 33(13), 1530-1532 (2008); examples of the Correlation Mapping OCT are disclosed by Enfield et al. “In vivo imaging of the microcirculation of the volar forearm using correlation mapping optical coherence tomography (cmOCT)” Biomed. Opt. Express 2, 1184-1193 (2011); examples of the OMAG are disclosed by An et al. “In vivo volumetric imaging of vascular perfusion within human retina and choroids with optical micro-angiography” Opt. Express 16, 11438-11452 (2008); examples of the Power Doppler OCT are disclosed by Makita et al. “Optical coherence angiography” Opt. Express 14, 7821-7840 (2006); examples of the SSADA are disclosed by Jia et al. “Split-spectrum amplitude-decorrelation angiography with optical coherence tomography,” Opt. Express 20(4), 4710-4725 (2012). The entire contents of these disclosures are incorporated herein by reference.

The OCT system for health characterization of an eye may comprise a generalized OCT system. For example, the OCT system may comprise at least one light source that provides the beam of light; at least one retro-reflector; at least one optical fiber coupler or at least one free space coupler that guides the beam of light to the physical object and to the at least one retro-reflector, wherein the beam of light guided to the physical object forms at least one backscattered light beam, and wherein the beam of light guided to the at least one retro-reflector forms at least one reflected reference light beam; at least one scanning optic that scans the at least one light beam over the physical object; and at least one detector. The at least one detector may combine the at least one backscattered light beam and the at least one reflected light beam to form light interference, detect magnitude and time delay of the at least one backscattered light beam, and forms at least one OCT signal. The at least one optical fiber coupler or the at least one free space coupler may guide the at least one backscattered light beam and the at least one reflected light beam to the at least one detector. The OCT system may further comprise at least one processor that obtains and analyzes the at least one OCT signal formed by the at least one detector, and forms an image of the physical object. The OCT system may also further comprise at least one display that displays the image of the physical object.

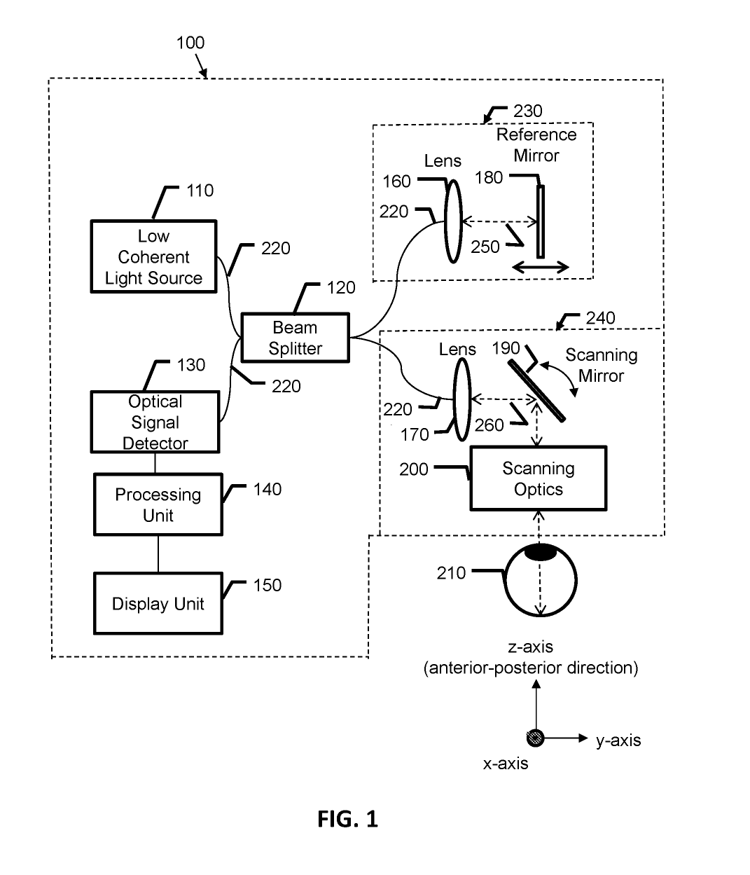

Examples of a generalized OCT system schematically shown in FIG. 1 are disclosed by Fingler et al. “Dynamic Motion Contrast and Transverse Flow Estimation Using Optical Coherence Tomography” U.S. Pat. No. 7,995,814; Fingler et al. “Dynamic Motion Contrast and Transverse Flow Estimation Using Optical Coherence Tomography” U.S. Pat. No. 8,369,594; and Sharma et al. in a U.S. Pat. No. 8,857,988, entitled “Data Acquisition Methods for Reduced Motion Artifacts and Applications in OCT Angiography”. These disclosures are incorporated herein by reference in their entirety. The OCT system 100 may comprise this generalized OCT system.

The OCT system 100 may comprise at least one light source 110, at least one scanning optic 200, at least one retro-reflector 180, at least one optical fiber coupler 220 or at least one free space coupler, at least one detector 130, at least one processing unit 140, and at least one display unit 150. The OCT system may further comprise a scanning mirror 190.

The at least one light source 110 may comprise any light source, for example, a low coherent light source. Light from the light source 110 may be guided, typically by using at least one optical fiber coupler 220 to illuminate a physical object 210. An example of the physical object 210 may be any tissue in a human eye. For example, the tissue may be a retina. The light source 110 may be either a broadband low coherence light source with short temporal coherence length in the case of SD-OCT or a wavelength tunable laser source in the case of SS-OCT. The light may be scanned, typically with the scanning optic 200 between the output of the optical fiber coupler 220 and the physical object 210, so that a beam of light (dashed line) guided for the physical object 210 is scanned laterally (in x-axis and/or y-axis) over the area or volume to be imaged. The scanning optic 200 may comprise any optical element suitable for scanning. The scanning optic 200 may comprise at least one component. The at least one component of the scanning optic 200 may be an optical component. Light scattered from the physical object 210 may be collected, typically into the same optical fiber coupler 220 used to guide the light for the illumination of the physical object 210. (The physical object 210 is shown in FIG. 1 only to schematically demonstrate the physical object 210 in relation to the OCT system 100. The physical object 210 is not a component of the OCT system 100.)

The OCT system 100 may further comprise a beam splitter 120 to split and guide the light provided by the light source 110 to a reference arm 230 and a physical object arm 240. The OCT system may also further comprise a lens 160 placed between the beam splitter 120 and the retro-reflector 180. The OCT system may also further comprise another lens 170 placed between the beam splitter 120 and the scanning optic 200.

Reference light 250 derived from the same light source 110 may travel a separate path, in this case involving the optical fiber coupler 220 and the retro-reflector 180 with an adjustable optical delay. The retro-reflector 180 may comprise at least one component. The at least one component of the retro-reflector 180 may be an optical component, for example, a reference mirror. A transmissive reference path may also be used and the adjustable delay may be placed in the physical object arm 240 or the reference arm 230 of the OCT system 100.

Collected light 260 scattered from the physical object 210 may be combined with reference light 250, typically in the fiber coupler to form light interference in the detector 130. Although a single optical fiber port is shown going to the detector 130, various designs of interferometers may be used for balanced or unbalanced detection of the interference signal for SS-OCT or a spectrometer detector for SD-OCT.

The output from the detector 130 may be supplied to the processing unit 140. Results may be stored in the processing unit 140 or displayed on the display unit 150. The processing and storing functions may be localized within the OCT system or functions may be performed on an external processing unit to which the collected data is transferred. This external unit may be dedicated to data processing or perform other tasks that are quite general and not dedicated to the OCT system.

Light beam as used herein should be interpreted as any carefully directed light path. In time-domain systems, the reference arm 230 may need to have a tunable optical delay to generate interference. Balanced detection systems may typically be used in TD-OCT and SS-OCT systems, while spectrometers may be used at the detection port for SD-OCT systems.

The interference may cause the intensity of the interfered light to vary across the spectrum. The Fourier transform of the interference light may reveal the profile of scattering intensities at different path lengths, and therefore scattering as a function of depth (z-axis direction) in the physical object. See for example Leitgeb et al. “Ultrahigh resolution Fourier domain optical coherence tomography,” Optics Express 12(10):2156, 2004. The entire content of this publication is incorporated herein by reference.

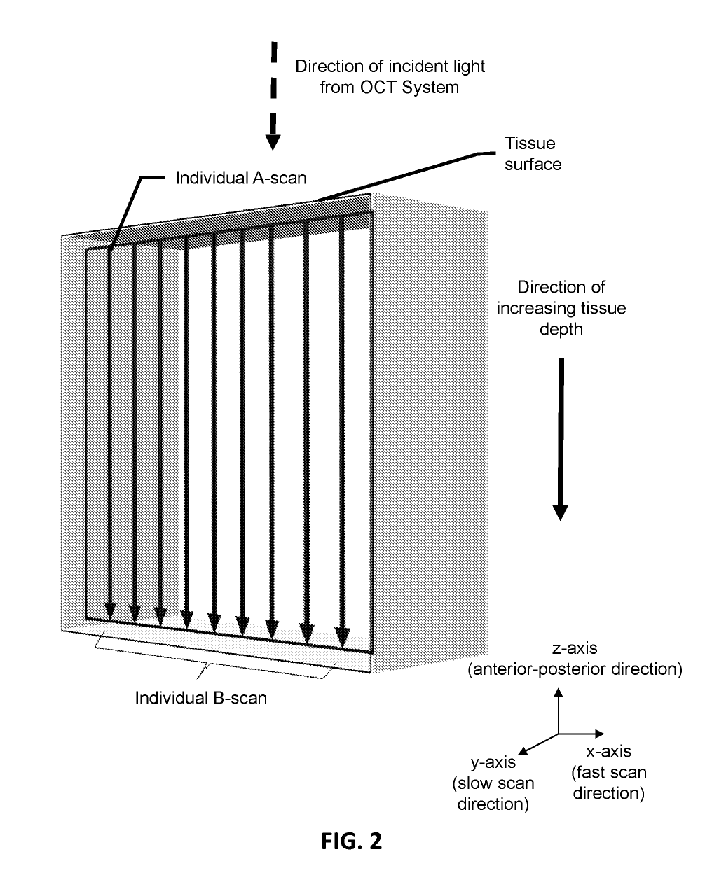

The profile of scattering as a function of depth is called an axial scan (A-scan), as schematically shown in FIG. 2. A set of A-scans measured at neighboring locations in the physical object produces a cross-sectional image (tomogram or B-scan) of the physical object. A collection of individual B-scans collected at different transverse locations on the sample makes up a data volume or cube. Three-dimensional C-scans can be formed by combining a plurality of B-scans. For a particular volume of data, the term fast axis refers to the scan direction along a single B-scan whereas slow axis refers to the axis along which multiple B-scans are collected.

B-scans may be formed by any transverse scanning in the plane designated by the x-axis and y-axis. B-scans may be formed, for example, along the horizontal or x-axis direction, along the vertical or y-axis direction, along the diagonal of x-axis and y-axis directions, in a circular or spiral pattern, and combinations thereof. The majority of the examples discussed herein may refer to B-scans in the x-z axis directions but this disclosure may apply equally to any cross sectional image.

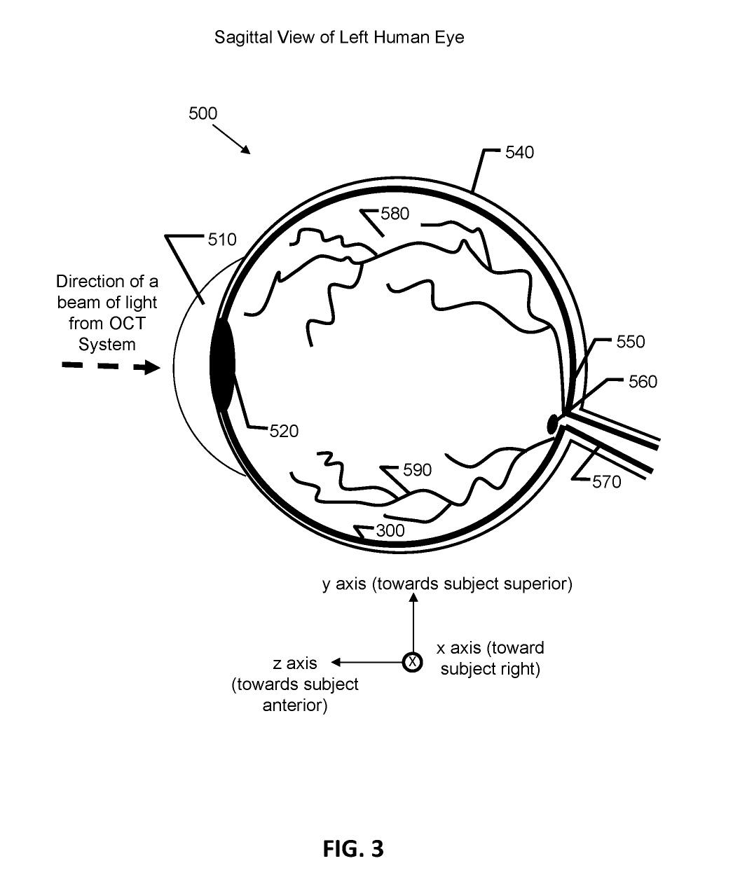

The physical object 210 may be any physical object. The physical object 210 may be a human eye, 500, as shown in a simplified manner in FIG. 3. The human eye comprises a cornea 510, a pupil 520, a retina 300, a choroid 540, a fovea region 550, an optic disk 560, an optic nerve 570, a vitreous chamber 580, and retinal blood vessels 590.

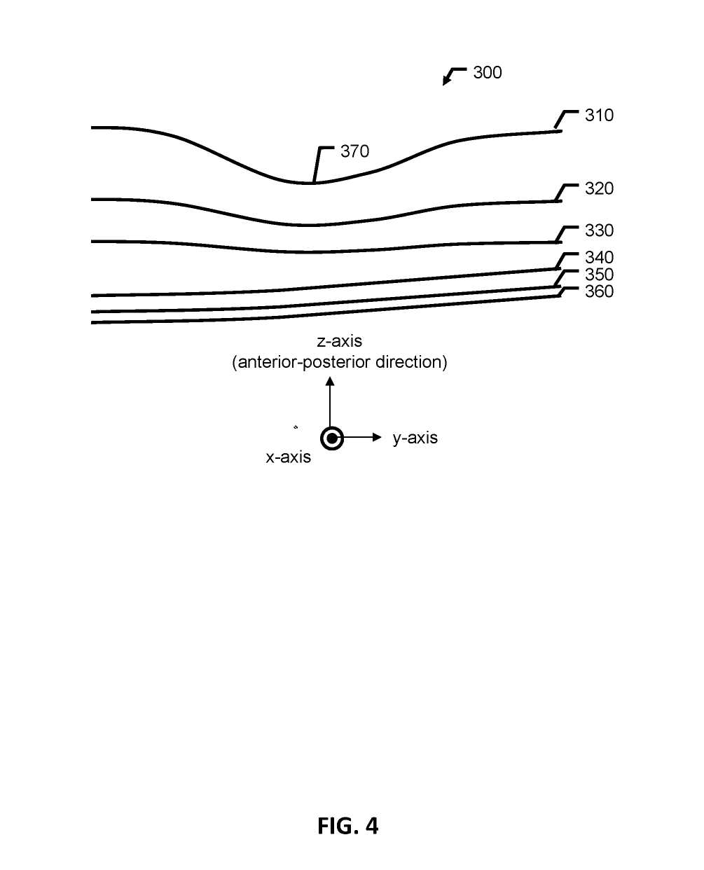

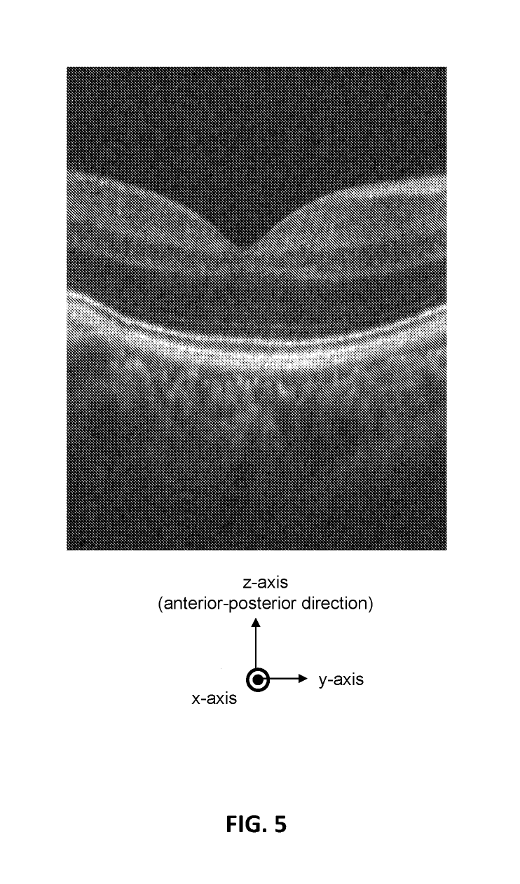

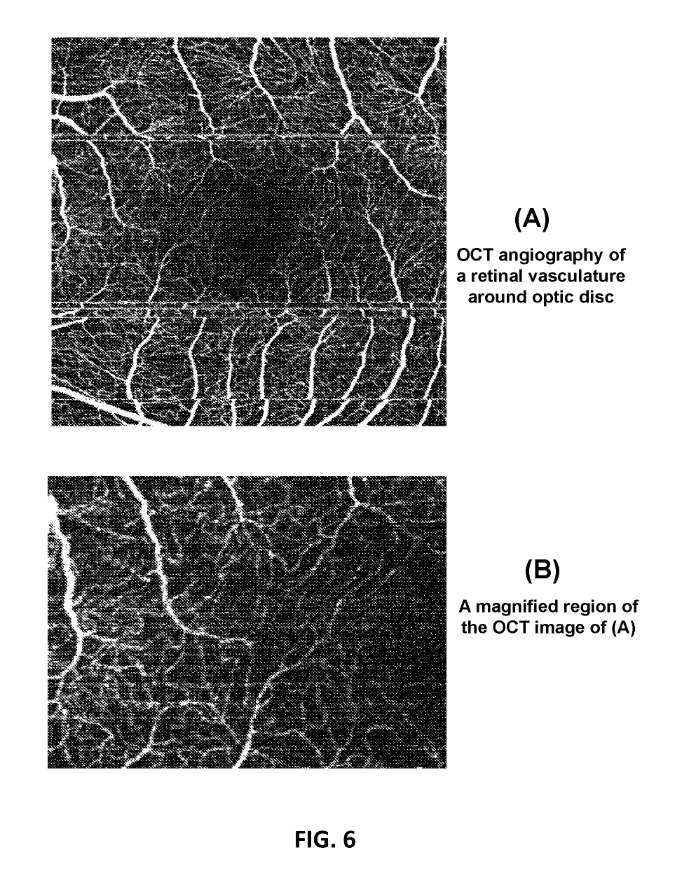

The physical object 210 may be tissue. An example of the tissue is a retina. A simplified cross-sectional image of layers of the retina 300 is schematically shown in FIG. 4. The retinal layers comprise a Nerve Fiber Layer (NFL) 310, External Limiting Membrane (ELM) 320, Inner/Outer Photoreceptor Segment 330, Outer Photoreceptor Segment 340, Retinal Pigment Epithelium (RPE) 350, Retinal Pigment Epithelium (RPE)/Bruch's Membrane Complex 360. FIG. 4 also schematically shows the fovea 370. FIG. 5 shows a cross-sectional OCT image of the fovea region of the retina. FIG. 6 shows (A) an exemplary en-face face OCT angiography image of a retinal vasculature around optic disc, (B) a magnified region of the OCT image of (A).

The physical object may comprise any physical object as disclosed above. The physical object has a surface and a depth. For example, a fundus of an eye has an outer surface receiving light from outside environment through the pupil. The fundus of an eye also has a depth starting at and extending from its outer surface.

In this disclosure, a z-axis (“axial axis”) is an axis parallel to the beam of light extending into the depth of the physical object, the x-axis and the y-axis (“transverse axes”) are transverse, thereby perpendicular axes to the z-axis. Orientation of these three axes is shown in FIGS. 1-5, 7 and 9.

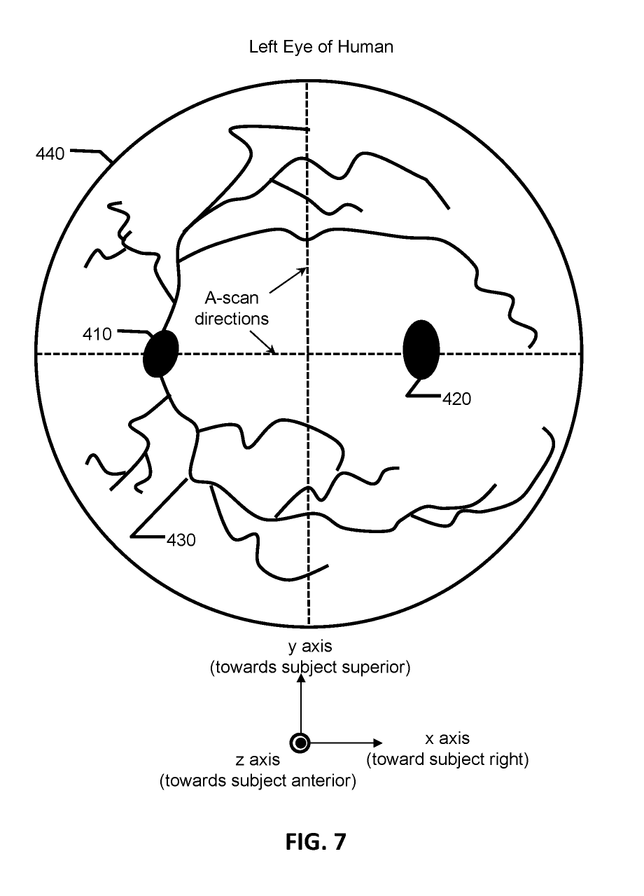

An example of the fundus of the eye is schematically shown in FIG. 7 in a simplified manner. In this circular visual field view 440 of the fundus of the eye, the anatomical landmarks are an optic disc 410, a fovea 420, and major blood vessels within the retina 430.

This disclosure relates to an extended duration optical coherence tomography (OCT) system for health characterization of an eye of a subject. The subject may be any mammal. The subject may be a human. The extended duration OCT system may include any OCT system disclosed above. The extended duration OCT system may have a configuration that (a) scans a tissue of the eye of a subject, which has a surface and a depth, with a beam of light that has a beam width and a direction; (b) acquires OCT signals from the scan; and (c) forms at least one B-scan cluster set using the acquired OCT signals.

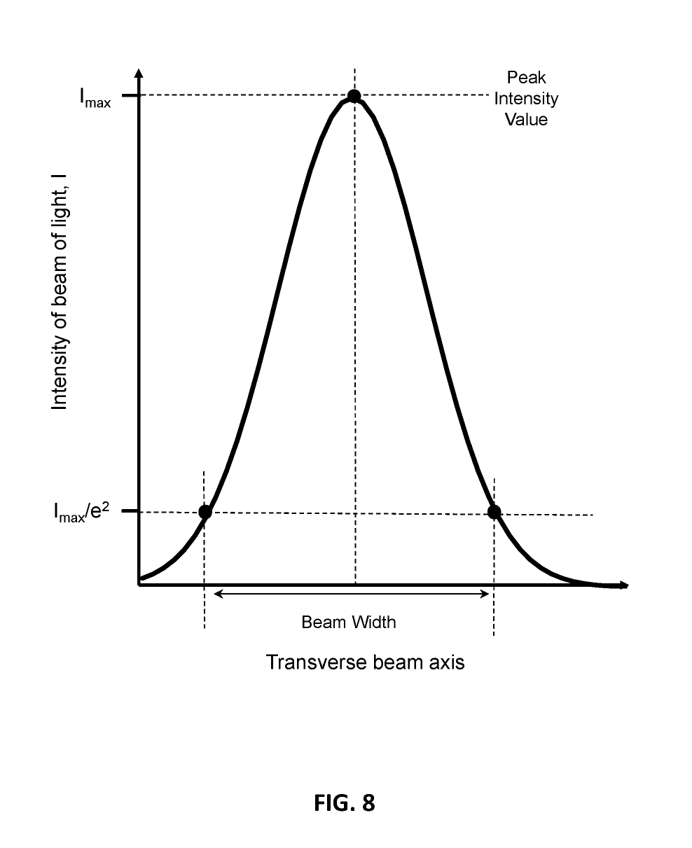

The beam of light provided by the OCT system has a width and an intensity at a location of the tissue of an eye. An example of the beam width is schematically shown in FIG. 8. This location may be at the surface of the tissue or within the tissue. In one example, at this location of the tissue, the beam of light may be focused (“focused beam of light”). For example, at this location the width of the beam of light may be at its smallest value. Cross-sectional area of the light beam may have any shape. For example, the cross-sectional area may have circular shape or elliptic shape. The intensity of the focused beam of light varies along its transverse axis, which is perpendicular to its propagation axis. This transverse beam axis may be a radial axis. The light beam intensity at the center of the light beam is at its peak value, i.e. the beam intensity is at its maximum, and decreases along its transverse axis, forming an intensity distribution. This distribution may be approximated by a Gaussian function, as shown in FIG. 8. The width of the beam of light (“beam width”) is defined as a length of line that intersects the intensity distribution at two opposite points at which the intensity is 1/e² times of its peak value. The light beam may comprise more than one peak. The peak with highest beam intensity is used to calculate the beam width. The beam width may be the focused beam of light. A typical beam width of a typical OCT system may vary in the range of 10 micrometers to 30 micrometers at the tissue location.

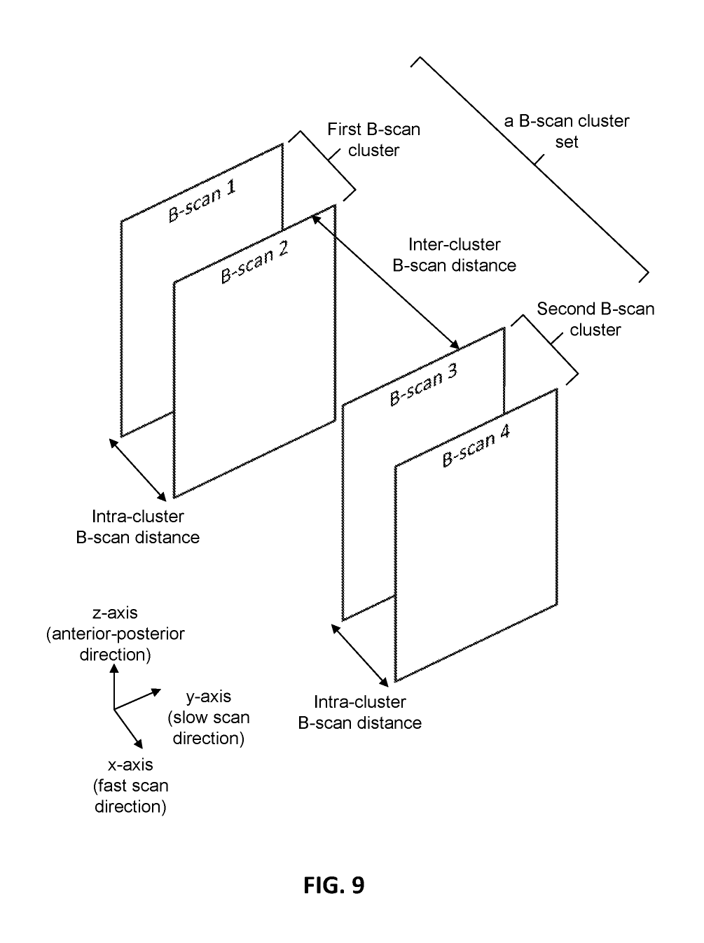

Each B-scan cluster set may include at least two B-scan clusters. Each B-scan cluster may include at least two B-scans. Each B-scan may include at least two A-scans. Each B-scan cluster set may be parallel to one another and parallel to the direction of the beam of light. The B-scans within each B-scan cluster set may be parallel to one another and parallel to the direction of the beam of light. An example of this system, shown in FIG. 9, comprises one B-scan cluster set comprising two B-scan clusters. And each B-scan cluster comprises two B-scans.

The extended duration OCT system may have a configuration to form more than one B-scan cluster. That is, a number of B-scan cluster set, P may be equal to or larger than 1, wherein P is an integer. For example, P may be 1, 2, 3, 4, 5, 10, 100, 1,000, 10,000, or 100,000.

Each B-scan cluster set may comprise any number of B-scan clusters, N equal to or greater than 2, wherein N is an integer. For example, N may be 2, 3, 4, 5, 10, 100, 1,000, 10,000, or 100,000.

Each B-scan cluster may comprise any number of B-scans, M equal to or greater than 2, wherein M is an integer. For example, M may be 2, 3, 4, 5, 10, 20, 100, 1,000, 10,000, or 100,000.

Each B-scan may comprise any number of A-scans, Q equal to or greater than 2, wherein M is an integer. For example, M may be 2, 3, 4, 5, 10, 20, 100, 1,000, 10,000, or 100,000.

Each A-scan, each B-scan, each B-scan cluster, and each B-scan cluster set may be acquired over a period of time. That is each A-scan, each B-scan, each B-scan cluster, and each B-scan cluster set may be formed at a different time than all other A-scans, B-scans, B-scan clusters, and B-scan cluster sets, respectively. In this disclosure, “first formed” means first formed in time; “next formed” means next formed in time; and “last formed” means last formed in time.

Each A-scan may be separated from any next A-scan by a distance (“A-scan distance”). The A-scan distance may be 0, at least 1 micrometer, or at least 10 micrometers.

Each B-scan within each B-scan cluster may be separated from any next formed B-scan within that B-scan cluster by a distance (“intra-cluster distance”) in the range of 0 to half of the beam width. For example, the intra-cluster distance may vary in the range of 0 to 15 micrometers.

The last formed B-scan within each B-scan cluster may be separated from the first formed B-scan within any next formed B-scan cluster (“inter-cluster distance”) by at least one micrometer. For example, the intra-cluster distance may vary in the range of 1 micrometer to 10 micrometers, 1 micrometer to 100 micrometers, or 1 micrometer to 1,000 micrometers.

The extended duration OCT system may have a configuration that calculates an OCT data using the at least one B-scan cluster. The OCT data may be an OCT angiography data that is calculated by using the at least one B-scan cluster and motion occurring within the eye tissue. The OCT angiography data may be calculated by using variations of intensity and/or phase of the OCT signals. This calculation may provide contrast. These variations may be variations caused by flow, speckle, and/or decorrelation of the OCT signal caused by eye tissue motion and/or flow in blood vessels of the eye tissue.

The extended duration OCT system may comprise a stabilized head positioning system, a dynamic fixation target system, a system for detecting blinking, an eye motion tracking system, a real-time data streaming and/or processing, a small and quick volumetric scanning, a system to block light to the non-imaged eye, or combinations thereof.

The extended duration OCT system may comprise a gravity-assisted head stabilization system. This system may be suitable to obtain high quality images. Most commercial OCT systems use a form of head and chin rest mount, wherein head and eye position stability is dependent on numerous factors such as chin and jaw stability, as well as the amount of pressure being applied by the forehead on the head rest, and thereby limiting the stabilization capabilities. Because of the lack of stability with these types of head mounts, many research labs use bite bars for stabilization, but this method may not be convenient enough for general purpose usage.

A head and/or body stabilization system that utilizes gravity to apply the required pressure on the subject (“gravity-assisted head stabilization system”) to create the positional stability desired by many types of ocular imaging systems, for example, OCT systems. Examples of the gravity-assisted head stabilization system include a tilted headrest system, an inclined chair system, a horizontal table system, or combinations thereof.

The tilted headrest system may comprise a rest for the forehead and cheekbones, oriented such that a seated subject only needs to look downward at a comfortable angle (to avoid neck strain) into the head rest, which is attached to the OCT system.

Examples of such systems are disclosed in connection with other ocular imaging systems. The extended duration OCT system may comprise such tilted headrest systems to improve head and eye stability. For example, a tilted headrest system is disclosed in connection with the Artemis VHF digital ultrasound arc scanner (Ultralink LLC, St. Petersburg, Fla.). This is an ocular imaging system for obtaining accurate measurements of the anterior segment for the management of myopes requiring correction with a phakic lens. See, for example, Roholt “Sizing the Visian ICL” Cataract and Refractive Surgery Today, May 2007. The entire content of this publication is incorporated herein by reference. In this system, the subject looks downward at approximately 45 degrees from vertical. The subject's head is positioned by a fixed chin rest and two fixed forehead rests that are adjusted mechanically to best position the subject's head. Heidelberg Engineering, Inc. (Heidelberg, Germany) has a similar tilted headrest system for their confocal scanning laser ophthalmoscope system used for corneal imaging. See, for example, the brochure “HRT Rostock Cornea Module” published by Heidelberg Engineering, Inc. The entire content of this publication is incorporated herein by reference. The OCULUS Easyfield C (Oculus, Inc., Arlington, Wash.) designed for use as a visual field screener also has a similar tilted headrest system wherein the subject looks downward at an angle from the vertical varying in the range of 31 degrees to 51 degrees. See, for example, the technical data available for OCULUS Easyfield C. The entire content of this publication is incorporated herein by reference. These exemplary tilted headrest systems may be suitable in providing required head and eye stability for the extended duration OCT system.

The inclined chair system may comprise a subject support system similar in concept to a massage chair, which may use an inclined design to stabilize the subject's head and body with gravity at a forward or a backward angle from the vertical. The head mount may be designed to maintain comfort for this position, while achieving enough clearance for imaging with the extended duration OCT system. For example, the head mount may comprise cushions to maintain comfort. Such cushions are disclosed, for example, by Eilers et al. in a U.S. Pat. No. 8,732,878, entitled “Method of Positioning a Patient for Medical Procedures”. The entire content of this disclosure is incorporated herein by reference.

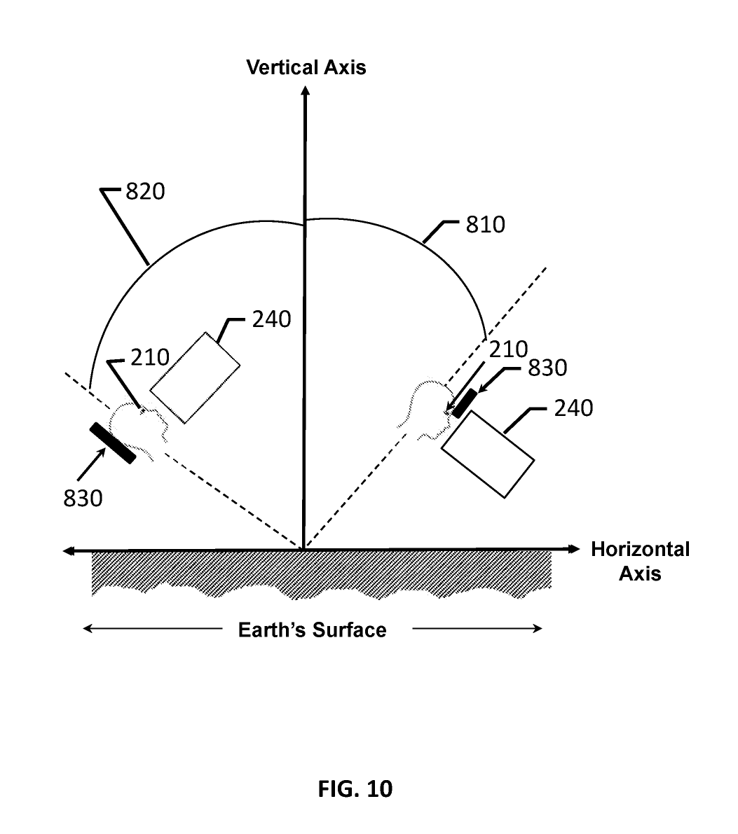

The tilted headrest system or the inclined chair system may comprise a headrest having a configuration such that when the subject rests his/her head on the headrest, the subject's head may be positioned at an angle with respect to an axis vertical to earth's surface. The subject may be a human. A simplified exemplary configuration is shown in FIG. 10. In this figure, the subject's head rests on the headrest 830 and the subject looks downward or upward towards the scanning optics of the OCT system 100 at an angle 810 or 820 (“tilt angle”) with respect to an axis perpendicular to earth's surface at the subject's location (“vertical axis”). This is an angle between the vertical axis and the axis passing through the subject' s cranial vertex (“vertex axis”), wherein the axis passing through the subject's cranial vertex is parallel to the subject's coronal plane. The tilt angle may be a positive angle 810. The positive angle 810 may be in the range of 10 degrees to 90 degrees. The positive angle 810 may also be in the range of 80 degrees to 90 degrees. The tilt angle may also be a negative angle 820. The negative angle 820 may be in the range of −10 degrees to −90 degrees. The negative angle 820 may also be in the range of −80 degrees to −90 degrees.

The horizontal table system may comprise a subject support system similar in concept to a horizontal massage table to stabilize the subject with gravity. The subject may be positioned on the horizontal table for forward viewing from face up or down position. The head mount may be designed to maintain comfort for this position, while achieving enough clearance for imaging with the ocular imaging system. At this configuration, the subject's head may be positioned at a tilt angle substantially close to 90 degrees or −90 degrees. At this configuration, the subject's head may be positioned at a tilt angle of 90 degrees or −90 degrees. At such position the vertex axis may be substantially parallel to the horizontal axis or parallel to the horizontal axis.

The extended duration OCT system may also comprise a dynamic fixation target system. The eye movement may be stabilized by having the subject focus on a target during the OCT imaging. Suitable examples of such dynamic fixation target systems and methods may comprise those used for the laser surgery of the subject eye's for variety of treatments. Examples of such systems may comprise a light emitting diode (LED) that may be optically positioned in front of or above the subject.

Heitel et al., in a U.S. Patent Application Publication No. 2014/0218689, entitled “Systems and Methods for Dynamic Patient Fixation System” discloses an eye fixation system that causes the eye to be fixated at a desired position, and an eye fixation adjustment system that enables the eye fixation system to be dynamically adjusted. This visual fixation system allows a subject's eye(s) to be accurately focused at one or more fixation targets. This patent application publication is incorporated herein by reference in its entirety. This system and method are suitable in providing eye stability for the extended duration OCT system.

Todd et al., in a U.S. Pat. No. 7,748,846, entitled “Dynamic Fixation Stimuli for Visual Field Testing and Therapy” discloses a system and a method wherein alteration of a fixation stimulus displayed on a computer-driven display allows a human subject to maintain extended visual fixation upon the resulting dynamic stimulus. In this disclosure, the fixation is presented upon the display and the stimulus is altered to allow resensitization of the subject's retina, thereby allowing prolonged visual fixation upon the resulting dynamic target. This patent is incorporated herein by reference in its entirety. This system and method is suitable in providing eye stability for the extended duration OCT system.

The extended duration OCT system may comprise a system for detection of blinking and compensating effects of blinking. The blinking is a semi-autonomic rapid closing of the eyelid. The effects of blinking may need to be minimized or entirely eliminated to obtain a wide field of view image of the retina suitable for angiography. The systems and/or methods have been proposed to minimize blinking effects as follows. These systems and/or methods may provide a system for detection of blinking and compensating effects of blinking and thereby they are within the scope of this disclosure. For example, see Narasimha-Iyer et al. “Systems and Methods for Improved Acquisition of Ophthalmic Optical Coherence Tomography Data” U.S. Patent Application Publication No. 2014/0268046. This disclosure is incorporated herein by reference in its entirety.

OCT instrument operators often ask the patient to blink once or twice before they start acquisition of data. Often times, however, the operator does not immediately recognize the blinking or take an unnecessarily long time to determine if the image quality and alignment is as good as before the blinking. This increases the time between blinking and start of acquisition and leaves less time before the subject is likely to blink or move again. Therefore the subject is more likely to blink or move again during the acquisition. In this disclosure, the system may automatically detect blinking of the subject, and starts the acquisition automatically, minimizing the time during which the patient has to stare into the device without blinking.

In order to reduce the often unnecessarily long time between blinking of the subject and start of the OCT acquisition, the extended duration OCT system may have a configuration that detects, for example, the double blinking of the subject, and then automatically starts acquiring data. Since blinking may block the light going into the eye and therefore directly results in OCT signal loss from e.g. the retina, the blinking may easily be detectable using optical techniques by looking for a strong instantaneous decrease or increase in optical signal or intensity. This may be accomplished using unprocessed or processed OCT data. One example may be analyzing the intensity of a series of fundus images generated from the OCT data in real time using a technique as described by Knighton in U.S. Pat. No. 7,301,644; entitled “Enhanced optical coherence tomography for anatomical mapping,” hereby incorporated by reference in its entirety. Alternatively, a stream of images from an adjunct camera, like an Iris Viewer as described by Everett in US Patent Publication No. 2007/0291277; entitled “Spectral domain optical coherence tomography system,” hereby incorporated by reference in its entirety, may be analyzed to detect when the eye is closed while blinking. In order to assure that the alignment is maintained after the blinking, the device may correlate the scans before and after the blinking. If sufficient correlation is achieved, the device may automatically start the acquisition. Such an automatic start of image acquisition would reduce the time the subject has to try not to blink and therefore ultimately improves patient comfort.

This system may further have a configuration that automatically stops acquisition of the OCT signals at onset of a blinking. This system may also further have a configuration that automatically starts acquisition of the OCT signals after a blinking. Also, this system may further have a configuration that automatically stops acquisition of the OCT signals at the onset of a blinking and automatically restarts acquisition of the OCT signals after the blinking stops.

The extended duration OCT system may comprise an eye motion tracking system and/or method to obtain high quality images. The eye motion tracking system and/or method may be any suitable eye motion tracking system and/or method that minimizes or prevents distortions caused by eye motion during acquisitions of the OCT scans.

For example, multiple B-scans (at the same location or closely spaced) may be obtained and analyzed to determine the change in the OCT data caused by motion. The extended duration OCT system and/or method may comprise such method.

Another example is disclosed by Sharma et al. in a U.S. Pat. No. 8,857,988, entitled “Data Acquisition Methods for Reduced Motion Artifacts and Applications in OCT Angiography”, which is incorporated herein by reference in its entirety. Sharma et al. propose a method, wherein two or more OCT A-scans may be obtained at the same location while the eye position is being monitored using tracking methods. With the use of eye tracking information, it may be ensured that at least two or more A-scans are obtained from the same tissue location, and the difference between the two A-scans is calculated and analyzed to ascertain structural or functional changes accurately without any eye motion related artifacts. The extended duration OCT system and/or method may comprise such systems and methods.

The extended duration OCT system may comprise a system and/or method for blocking light to the non-imaged eye. The non-imaged eye may be blocked to minimize or avoid additional fixations issues or distractions that may be caused for the unblocked non-imaged eye.

The OCT motion contrast method disclosed above may be used for any OCT related application. For example, this method maybe used in forming larger field of view OCT images of the physical object. This method may be incorporated into methods and systems related to OCT based angiography. For example, the choroidal vasculature may be identified in more detail by using the OCT motion contrast method. The OCT methods comprising the OCT motion contrast method also be used in diagnosis and/or treatment of health conditions such as diseases. For example, the OCT methods comprising the OCT motion contrast method may be used in characterization of retinal health.

The OCT system disclosed above may provide any information related to the physical object. For example, this system, which may uses the motion contrast method, may provide 2D (i.e. cross-sectional) images, en-face images, 3-D images, metrics related to a health condition, and the like. This system may be used with any other system. For example, the OCT system may be used with an ultrasound device, or a surgical system for diagnostic or treatment purposes. The OCT system may be used to analyze any physical object. For example, the OCT system may be used in analysis, e.g. formation of images, of, for example, any type of life forms and inanimate objects. Examples of life forms may be animals, plants, cells or the like.

Unless otherwise indicated, the processing unit 140 that has been discussed herein may be implemented with a computer system configured to perform the functions that have been described herein for this unit. The computer system includes one or more processors, tangible memories (e.g., random access memories (RAMs), read-only memories (ROMs), and/or programmable read only memories (PROMS)), tangible storage devices (e.g., hard disk drives, CD/DVD drives, and/or flash memories), system buses, video processing components, network communication components, input/output ports, and/or user interface devices (e.g., keyboards, pointing devices, displays, microphones, sound reproduction systems, and/or touch screens).

The computer system for the processing unit 140 may include one or more computers at the same or different locations. When at different locations, the computers may be configured to communicate with one another through a wired and/or wireless network communication system.

The computer system may include software (e.g., one or more operating systems, device drivers, application programs, and/or communication programs). When software is included, the software includes programming instructions and may include associated data and libraries. When included, the programming instructions are configured to implement one or more algorithms that implement one or more of the functions of the computer system, as recited herein. The description of each function that is performed by each computer system also constitutes a description of the algorithm(s) that performs that function.

The software may be stored on or in one or more non-transitory, tangible storage devices, such as one or more hard disk drives, CDs, DVDs, and/or flash memories. The software may be in source code and/or object code format. Associated data may be stored in any type of volatile and/or non-volatile memory. The software may be loaded into a non-transitory memory and executed by one or more processors.

Any combination of methods, devices, systems, and features disclosed above are within the scope of this disclosure.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

In this disclosure, the indefinite article “a” and phrases “one or more” and “at least one” are synonymous and mean “at least one”.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. An extended duration optical coherence tomography (OCT) system for health characterization of an eye of a human subject that comprises:
an OCT data acquisition system having a configuration that:
scans tissue of an eye of a subject, which has a surface and a depth, with a beam of light that has a beam width and a direction;
acquires OCT signals from the scan;
forms at least one B-scan cluster set using the acquired OCT signals such that each B-scan cluster set includes at least two B-scan clusters; each B-scan cluster includes at least two B-scans; and each B-scan includes at least two A-scans; and
calculates OCT data using the at least one B-scan cluster set; and
a gravity-assisted head stabilization system that provides stability for the subject's head and the eye when the OCT data acquisition system scans the tissue,
wherein the gravity-assisted head stabilization system comprises a headrest; and
wherein the headrest has a configuration such that, when the subject rests his/her head on the headrest, an axis passing through the subject's cranial vertex and that is parallel to the subject's coronal plane (“vertex axis”) is not perpendicular to the surface of the earth at the location of the subject.

2. The extended duration OCT system of claim 1, wherein the headrest has a configuration such that when the subject rests his/her head on the headrest, the angle (“tilt angle”) between the vertex axis and an axis perpendicular to the surface of the earth at the location of the subject (“vertical axis”) is in the range of 10 degrees to 90 degrees.

3. The extended duration OCT system of claim 1, wherein the headrest having a configuration such that when the subject rests his/her head on the headrest, the angle (“tilt angle”) between the vertex axis and an axis perpendicular to the surface of the earth at the location of the subject (“vertical axis”) is in the range of −10 degrees to −90 degrees.

4. The extended duration OCT system of claim 2, wherein the tilt angle is in the range of 80 degrees to 90 degrees.

5. The extended duration OCT system of claim 3, wherein the tilt angle is in the range of −80 degrees to −90 degrees.

6. The extended duration OCT system of claim 1, wherein the OCT data acquisition system comprises a physical object arm; and wherein the physical object arm is mechanically affixed to the headrest.

7. The extended duration OCT system of claim 1, wherein the gravity-assisted head stabilization system further comprises an inclined chair system, a horizontal table system, or a combination thereof.

8. The extended duration OCT system of claim 1, wherein the system further comprises a dynamic fixation target system that stabilizes movement of the subject's eye; and wherein the dynamic fixation target system comprises at least one fixation target.

9. The extended duration OCT system of claim 1, wherein the system further comprises a system that automatically detects blinking of the subject and compensates for effects of blinking on the calculated OCT data.

10. The extended duration OCT system of claim 9, further having a configuration that automatically stops acquisition of the OCT signals at onset of a blinking.

11. The extended duration OCT system of claim 9, further having a configuration that automatically starts acquisition of the OCT signals after a blinking.

12. The extended duration OCT system of claim 9, further having a configuration that automatically detects blinking by detecting a strong instantaneous decrease or increase in intensity of the acquired OCT signals.

13. The extended duration OCT system of claim 9, further having a configuration that automatically detects blinking by using the calculated OCT data.

14. The extended duration OCT system of claim 9, wherein the system further comprises a camera; and the system further has a configuration that uses images provided by the camera to detect blinking.

15. The extended duration OCT system of claim 1, wherein the system further comprises an eye motion tracking system and uses information provided by this tracking system to minimize effects of the eye motion on the calculated OCT data.

16. The extended duration OCT system of claim 1, further having a configuration that blocks light to a non-imaged eye.

17. The extended duration OCT system of claim 1, further having a configuration that calculates OCT data using motion occurring within the eye tissue and the at least one B-scan cluster set.

18. The system of claim 17, wherein the OCT data is calculated by using variations of intensity or phase of the OCT signals to provide contrast.

19. The system of claim 18, wherein the OCT data is calculated by using variations of intensity or phase of the OCT signals caused by flow, speckle, or decorrelation of an OCT signal within the OCT signals that is caused by eye tissue motion or blood flow in blood vessels of the eye tissue.
